(19) United States
(12) Patent Application Publication
(10) Pub. No.: US 2013/0103079 A1
(43) Pub. Date: Apr. 25, 2013

(54) BIOMIMETIC MULTIPLE STRAND FIBER MESH AND SUTURES

(71) Applicant: Novo Contour, Inc., Edmonds, WA (US)

(72) Inventors: Michael P.H. Lau, Edmonds, WA (US); Leonard Pease, Bountiful, UT (US)

(73) Assignee: NOVO CONTOUR, INC., Edmonds, WA (US)

(21) Appl. No.: 13/659,861

(22) Filed: Oct. 24, 2012

Related U.S. Application Data
(60) Provisional application No. 61/550,693, filed on Oct. 24, 2011.

(51) Int. Cl. C08L 83/04; A61L 17/08; A61L 17/00

(52) U.S. Cl. 606/229; 606/228; 525/450

(57) ABSTRACT

A material comprising two or more fibers, wherein each of the fibers has a mechanical modulus, and the mechanical modulus of at least one fiber is higher than the mechanical modulus of another fiber. The higher modulus fiber has a longer length than the lower modulus fiber. In various embodiments, the higher modulus fiber is collagen mimetic and the lower modulus fiber is elastin mimetic. A suture is also described, comprising two or more fibers. At least one of the fibers is elastin-like and has a lower elastic modulus than another fiber that is collagen-like and has a higher elastic modulus. The higher modulus collagen-like fiber is longer than the lower modulus elastin-like fiber.

### CROSS-REFERENCE TO RELATED APPLICATION

This application claims the benefit of U.S. Provisional Patent Application No. 61/550,693, filed Oct. 24, 2011, the disclosure of which is incorporated by reference herein in its entirety.

### TECHNICAL FIELD

The present disclosure relates to a material, such as a biomimetic mesh, for use as a tissue support, tissue scaffold, tissue replacement, bandage element, suture, and/or as an element in surgical meshes and the like.

### BACKGROUND

Urinary incontinence and pelvic floor disorders adversely affect millions of women leading to embarrassment, incapacitating falls, and nursing home admission. In child bearing years, damage to fascia, a connective tissue, and muscles in the pelvic floor during delivery leads to stress urinary incontinence (SUI), a particular form of incontinence characterized by loss of urine upon jumping, coughing, sneezing, or other physical exertion. SUI incidence peaks during midlife and accounts for 55.4% of incontinence in the U.S. Later in life, incontinence leads to over 50% of nursing facility admissions, and pelvic organ prolapse becomes more significant as urogenital organs lose mass. Nearly 30 to 40% of American women suffer from some form of incontinence, and nearly one in six women will be affected by SUI over their lifetimes. Almost 10% of women will receive surgical treatment for urinary incontinence and pelvic organ prolapse at least once in their lifetime. Approximately 180,000 surgeries for SUI and one-half million surgeries for pelvic organ prolapse are performed each year in the U.S.

Genital prolapse, including cystocele, rectocele, enterocele, and uterine prolapse, along with stress incontinence, affect nearly one in four U.S. women (28.1 million), and by 2050, the number is projected to grow to nearly 44 million. Women, especially elderly women, often find this pelvic floor disorder too embarrassing to disclose. However, the effect of genital prolapse can be quite disabling. Prolapse of the vaginal wall and pelvic organs is due to weakening of the endopelvic fascia and supportive ligaments of the pelvis. The weakening is usually caused by childbirth, compounded by the aging process and occasionally trauma.

Surgical treatment for urinary incontinence involves the placement of either natural tissue or synthetic mesh to support the urethrovesicle junction (UVJ), commonly called the bladder neck. In healthy patients, the UVJ is adequately supported by a thin (˜3 mm thick) layer of pelvic fascia. When the fascia weakens or elongates over time or stretches due to childbirth, the UVJ falls from its preferred position superior to the base of the bladder during Valsalva's events, allowing urine loss. To provide additional support, most gynecological surgeons use polymer meshes that are readily available, sterilized, and inserted as a suburethral sling on an outpatient basis.

The repair of a prolapse involves the plication of the supportive endopelvic fascia or ligaments, using sutures, after extensive dissection of the pelvic structures along the tissue planes. In many women, especially the elderly, the supportive endopelvic fascia or ligaments are so thinned out or non-existent as to make suturing to plicate very challenging, if not impossible. Traditionally, autologous or donor tissue is used to supplement the deficient pelvic support tissue. Such use of biologic materials adds significant complexity and extensiveness of the surgery and has its associated risks. For these reasons, more surgeons are turning to the use of synthetic surgical mesh or acellular collagen network of porcine dermal material to augment the traditional genital prolapse repair. These materials have been increasingly included as part of commercially pre-packaged minimally invasive surgical kits for pelvic floor repair. The kits usually involve anchoring the mesh material to some fixed tissue points in the pelvis or relying on tissue-mesh friction to hold the mesh in place.

Despite their broad clinical acceptance, synthetic mesh has recently come under increased scrutiny from the FDA due to concerns over erosion. This complication occurs when the polymer mesh cuts through (i.e., erodes) adjacent tissue, penetrating the bladder, vagina, or urethra depending on initial placement. The eroded area causes loss of organ function and chronic discharge, becomes susceptible to infection, often causes painful rejection of the mesh, and requires surgical reintervention. In 1999, the FDA removed the worst offending meshes, those made of polyester, completely from the market. Since that time, polypropylene meshes have captured the greatest share of the market, though they also have erosion rates ranging from 3.4% to as high as 23%. Consequently, in late October 2008, the FDA warned of possible complications from all commercially available polymer meshes used for pelvic organ prolapse and stress urinary incontinence. A nearly unprecedented second warning was issued on Jul. 13, 2011. These warnings indicate a long-standing need for meshes that minimize erosion, which may be accomplished by more precisely mimicking, copying, duplicating, or imitating the properties of natural tissue.

Furthermore, continuous sheets used as fascial replacements are generally unacceptable for treatment of stress urinary incontinence and pelvic organ prolapse. Uniform sheets either spanning the full length of the fascia to be replaced or supported on fiber mesh are associated with higher rates of infection than fibers.

### SUMMARY

In various embodiments, the present application describes a material comprising two or more fibers. Each of the fibers has a mechanical modulus, wherein the mechanical modulus of at least one fiber is higher than the mechanical modulus of another fiber. The higher modulus fiber has a longer length than the lower modulus fiber. The higher modulus fiber may be collagen mimetic, while the lower modulus fiber may be elastin mimetic.

The fibers may be a monofilament fiber and/or a polyfilament fiber. In various embodiments, the lower modulus fiber may have an elastic modulus in the range of 0.1 to 10 MPa. The higher modulus fiber may have an elastic modulus in the range of 1 to 10000 MPa, and may be untensioned with a wavy configuration.

In various embodiments, the fibers may be woven or knitted to form a network. Additionally, the fibers may be biodegradable or non-biodegradable. Furthermore, the fibers may be arranged to produce an auxetic material in which the width of the auxetic material expands instead of shrinks upon tensile stress. In various embodiments, the fibers may be arranged to produce fabric sheets, and 2 to 200 fabric sheets are layered together.

Further described herein is a suture comprising two or more different fibers. At least one of the fibers is elastin-like and has a lower elastic modulus than another fiber that is collagen-like and has a higher elastic modulus. The higher modulus collagen-like fiber is longer than the lower modulus elastin-like fiber.

In at least one embodiment, the collagen-like fiber surrounds the elastin-like fiber. In another embodiment, the collagen-like fiber is positioned within a hollow elastin-like fiber.

This summary above is provided to introduce a selection of concepts in a simplified form that are further described below in the Detailed Description. It should be understood that this summary is not intended to identify key features of the claimed subject matter, nor is it intended to be used as an aid in determining the scope of the claimed subject matter.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a graph illustrating a comparison of rabbit deep tibial fascia to the polymers polyethylene (PE), polypropylene (PP), poly(glycerol sebacate) (PGS), wherein the star denotes the approximate critical stress;

FIG. 2 provides pictorial diagrams illustrating spatial arrangements of elastin (thin) and collagen (thick) fibers in (a) 1D and 2D with (b) elastin in a square lattice and loose collagen, (c) elastin in a square lattice and collagen in a zig-zag pattern (p=q=1), (d) elastin in a narrow diamond lattice and loose collagen, (e) elastin in a wide diamond lattice and loose collagen, and (f) elastin in a narrow diamond lattice and collagen in a zig-zag arrangement (p=q=1), and for (g) square and (h) diamond lattices, the nodes are numbered as ordered pairs (i, j) denoting row and column position, respectively, and for (i), crimped or zig-zag collagen pathways are shown with p=2 and q=1 (left) and p=1 and q=2 (right);

FIG. 3 provides graphs illustrating stress-strain or scaled force-displacement curves for (a) elastin and collagen-like fibers for Δu*/Lₒ=0 (-- - --), ½ (------) 1 (-- - -), 3/2 (-- -- --), and elastin only (- - -) with n_{cs}E_{c}A_{c}L^{o}/nₑₛEAL_{c}^{o}=100, and (b) elastin and collagen-like fibers for n_{cs}E_{c}A_{c}L^{o}/nₑₛEAL_{c}^{o}=1000 (-- - --), 100 (------) 10 (-- - -), 1 (-- -- --), with αu*/Lₒ=0.5, wherein insets show the nodal network under various degrees of strain;

FIG. 4 is a graph illustrating stress-strain curves for two 1D elastic materials with variable overhang of the higher modulus strand arranged in parallel to change the critical strain;

FIG. 5 is a graph illustrating experimental stress-strain data for bovine abdominal fascia;

FIG. 6 is a graph illustrating experimental stress-strain data for monkey abdominal fascia; and

FIG. 7 is a photograph of a dual strand mesh composed of PDMS representing elastin-like fibers and nylon strands representing collagen-like fibers.

### DETAILED DESCRIPTION

Embodiments of the present disclosure provide solutions to long-standing needs, for example, for a material that minimizes tissue erosion in the field of surgical treatment of organ prolapse, urinary incontinence, and related maladies. Embodiments of the disclosure further address the field of surgical sutures that, for example, enhance microcirculation to minimize tissue strangulation and wound edge necrosis.

Biomimetic solutions to these problems are preferential. Erosion is virtually unknown for tissue grafts presumably because the mechanical response of tissue is similar to that of the patient's fascia. This indicates that erosion is caused, at least in part, by a mismatch between the mechanical properties of the patient's fascia and the synthetic mesh, particularly the shear modulus. Most mesh materials do not give significantly, causing the tissue to kinetically slide along the surface of the mesh. Shear between tissue and mesh induces friction and heating that weaken the tissue, induce an immune response, and allow the polymer strands to erode through the tissue. Furthermore, postmenopausal women are particularly susceptible to erosion because their fascia and vaginal mucosa are particularly thin, exacerbating the difference in mechanical strength. Indeed, the worst offending meshes had to be removed from the market per FDA action. Tissue grafts are much softer and more giving than commercially available synthetic meshes.

It is recognized that biomaterials such as fascia and skin display nonlinear stress-strain relationships. Stress is the amount of force applied divided by the cross-sectional area of the material normal to that force. Strain is the change in displacement due to the force divided by the initial length of the material, where the change in displacement is the difference between the initial and current material length. At low stresses in the toe region, fascia elongates significantly as elastin fibers readily yield to the applied stress. This region of the stress-strain curve is called the toe or pre-transition region. At higher stresses, networked collagen fibers, initially crimped, straighten out to provide increased stiffness. This region of the stress-strain curve is called the linear region or the transition region. The slope of the curve is defined as the elastic or tensile modulus, E. The shear modulus, G, is defined as E/[2(1+v)], where v is Poisson's ratio. Poisson's ratio represents the ratio of the change in material length in one direction to that in another, typically the ratio of change in material length normal to the force divided by that parallel to the force. Clearly the shear modulus changes as a function of the strain. Because the toe and linear regions have different slopes, there exists a strain at which the transition between the two regions occurs. This strain is referred to as the critical strain, the transition strain, or the lock-up strain.

The source of this two-slope behavior is believed to arise from the combination collagen and elastin/fibrillin fibers that form connective networks within the extracellular matrix. The elastin and fibrillin fibrils deform easily with elastic moduli on the order of 0.01-10 MPa, whereas collagen fibrils bundled in various arrangements have elastic moduli on the order of 1-10000 MPa. To allow the elastin and fibrillin to govern (perhaps with partial participation of collagen) the tissue response at modest stresses, the collagen fibers remain limp. This is achieved in tendon fibers by arranging the collagen in wavy or “zig-zag” patterns that do not sustain significant amounts of stress until the individual fibers have rotated to align or aligned with the principle stress axis. Both in plane and out of plane zig-zag patterns are observed. Thereafter, the collagen fibers alone govern tissue response. Initially unstressed collagen fibers are a significant feature of fascia, as yet not duplicated in synthetic fascial mesh, particularly for urinary incontinence and pelvic organ prolapse. A stronger fiber allows for constructive remodeling and preserves functionality during repair while a weaker fiber provides a more biomimetic cushion minimizing erosion.

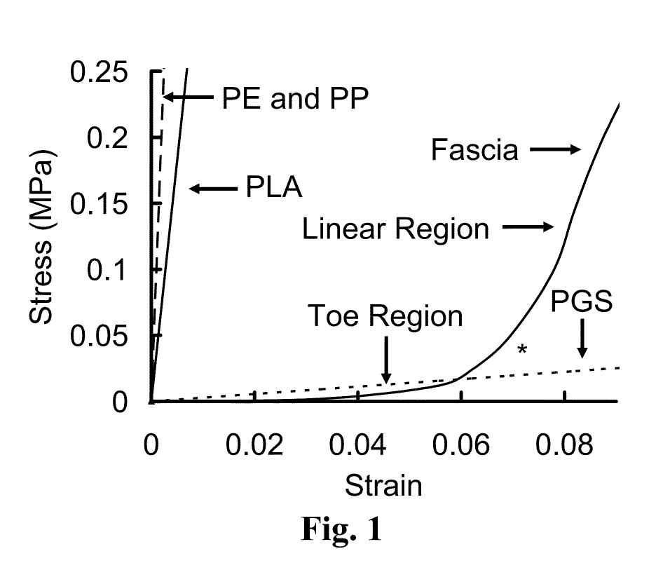

Indeed, synthetic polymers have linear elastic profiles with only a single value for the modulus and do not give significantly at low stresses (see FIG. 1). Indeed, no known fibrous implant precisely reproduces the mechanical properties of native tissue leading to serious consequences, particularly when implanted in mechanically active locations. No known combination of fibers or fiber mesh duplicates this multiple slope or strain dependent modulus behavior observed for fascia.

Monofilament fibers are associated with even lower rates of infection than polyfilament fibers. Therefore, fiber meshes are preferential to uniform sheets, films, or membranes, with or without embedded or coupled fibers for reinforcement. To minimize infections, open spaces within the mesh should exceed 10, 20, 30, 40, 50, 60, 70, 80, 90, 100, 110, 120, 130, 140, or 150 microns.

Embodiments of the disclosure address each of the above concerns to overcome long standing needs, particularly for the treatment of urinary incontinence and/or pelvic organ prolapse.

Furthermore, embodiments of the disclosure apply to sutures, bandages, and the like. Typical sutures and bandages are constructed out of a single material and therefore strain linearly with applied stress, and hence can be easily over tightened. Over tightening leads to puckering, wound edge necrosis, decreased microcirculation, and tissue strangulation. In each of these cases, designing some give into the suture while still maintaining the ability of the suture to hold tissue together is advantageous. In other words, sutures and bandages designed to give at low stresses but not at high stresses are advantageous. Embodiments of the disclosure provide a solution to this problem enhancing microcirculation, minimizing puckering, minimizing wound edge necrosis, and preventing tissue strangulation particularly for running locked sutures, pulley sutures, horizontal mattress sutures, 3-point corner stitches, inter alia.

In various embodiments, disclosed herein is an innovative biomimetic soft tissue replacement material comprising at least two fibers, one of which is an elastin-mimetic fiber and the other is a collagen-mimetic fiber.

In various embodiments, further disclosed herein is a fiber mesh comprising two or more fibers to approximate the mechanical properties of fascia. One fiber is more elastic similar to elastin, and the second fiber has properties approximating those of collagen. Elastin-like or elastin mimetic fibers are fibers that have relatively low mechanical moduli (which may be an elastic moduli, also referred to as tensile moduli or Young's moduli, the singular form of which is modulus), typically in the range of 0.1-10 MPa. Collagen-like fibers have relatively higher mechanical (e.g., elastic) moduli in the range of 1-10000 MPa. In a preferred embodiment, to develop a polymeric mesh with mechanical properties similar to those of intrinsic tissue, two or more types of fibers are networked together to mimic the network of elastin and collagen strands within the natural fascia, such as the endopelvic fascia that synergistically cradles the UVJ. Combining two or more fibers together provides both microstress transfer at the cellular level and tissue support at the organ/tissue level.

As disclosed herein, the fibers with the greater moduli (e.g., the collagen-like fibers) remain untensioned (i.e., loose or limp) until a critical strain is achieved at which point the fibers with the greater moduli engage and fully participate in bearing the stress. Preferred embodiments discussed below include a plurality of elastin-like fiber arrangements including but not limited to square or diamond lattices. The preferred embodiments below also disclose a plurality of spanning collagen-like fiber arrangements including but not limited to curvilinear and zig-zag arrangements. In each case, collagen-like fibers are left initially unstressed or at least partially unstressed. The plurality of fiber arrangements enables more precise mimicking, copying, duplicating, or designing of fiber arrangements and densities that match the material properties of bovine and human fascia.

As disclosed in the examples below, the unstressed collagen fibers may undulate perpendicular to the plane of the tissue, but surprisingly the model also indicates a variety of other configurations including undulating in-plane or simply remaining loose in random configurations without periodic or quasi-periodic waviness. Embodiments of the disclosure herein use monofilament fibers woven or formed to prevent infections in contrast to prior elastin-mimetic lamina, sheets, membranes, or films. Furthermore, the fibers disclosed herein may be broadly any biocompatible fiber including those generally recognized as safe (GRAS) by the FDA.

Furthermore, embodiments disclosed herein include biodegradable fiber meshes for use as a suburethral and vaginal slings. Biodegradable meshes are important because erosion often develops gradually over months and years, yet the patient's own fascia typically develops sufficient structural support within a few weeks to months following surgery. Mesh that remains after this initial recovery period seldom serves a useful purpose and may be harmful by causing erosion or generating scar tissue. Nearly 70% of erosion cases occur more than one year post surgery. A biodegradable mesh directly eliminates this risk. Biodegradable slings consisting of a solid sheet of biodegradable polymer were previously introduced under the name SABRE (Johnson & Johnson) but have not achieved significant market share because sheets predispose the patient to increased risk of infection relative to fiber meshes generally, and monofilament in particular. Here the first biodegradable monofilament fiber meshes for pelvic organ prolapse and urinary incontinence are disclosed.

In at least one preferred embodiment, the fibers are comprised of biocompatible materials, including but not limited to polypropylene and poly(dimethyl siloxane). In another preferred embodiment, the fibers are comprised of biodegradable materials, including but not limited to polyglycolic acid, polylactic acid, polydioxanone, and polycaprolactone and copolymers and blends thereof.

In another preferred embodiment, the mesh is comprised of monofilament strands produced using electrospun fibers of poly(lactic acid) (PLA) and poly(glycerol-sebacate) (PGS). These two polymers were chosen as examples because they have mechanical properties most closely approximating those of collagen and elastin, respectively. This is the first time that these two biodegradable/biocompatible materials have been used together in surgical meshes.

In another preferred embodiment, the mesh is comprised of monofilament strands of poly(lactic acid) (PLA) and poly(dimethyl siloxane) (PDMS) or poly(lactic acid-glycolic acid) (PLGA) and poly(dimethyl siloxane) (PDMS). These two polymers were chosen as examples because they have mechanical properties most closely approximating those of collagen and elastin, respectively. This is the first time that these two biodegradable/biocompatible materials have been used together in surgical meshes.

In another preferred embodiment, one or more of the fibers displays surface erosion properties critical to maintaining mechanical integrity during a gradual, well tuned degradation process. Classes of polymers that satisfy this requirement include polyanhydrides and polymers formed by polycondensation reactions. Embodiments of the disclosure herein include members of both classes. Additional classes of surface erodible polymers lie within the scope of the present disclosure.

In various embodiments, elastin-like fibers and collagen-like fibers may be used. In a preferred embodiment, the fibers are networked together by weaving, knitting, or other fiber manipulation techniques known to those skilled in the art. In one embodiment, the elastin-like fibers are formed into an elastic fabric, and the collagen-like fibers are formed into an elastic fabric with a higher modulus. In a preferred embodiment, the elastin-like fibers are woven into a square arrangement. In another preferred embodiment, the elastin-like fibers are woven into a diamond arrangement. Those skilled in the art will understand that a plethora of structural arrangements lie within the scope of the present disclosure. The fabrics are then layered together, for example, in 2 to 200 layers, with the collagen-like fiber remaining loose. In a preferred embodiment, the collagen-like fibers are arranged in a zig-zag, crimped, or wavy configurations. In a preferred embodiment, the fabrics are annealed or fixed together. In a preferred embodiment, the ends of the fabrics are merged together. In a preferred embodiment, the collagen-like fibers are simply loose or limp without preferred undulation. In at least one embodiment, the wavy configurations (e.g., the undulations, zig-zags, or crimps) are normal to the plane of the fabric. In a preferred embodiment, the wavy configurations are parallel to the plane of the fabric.

In a preferred embodiment, the fibers are interwoven or interlocking to form an interpenetrating network comprising a fabric. In a preferred embodiment, the elastin-like fibers are woven into a square arrangement. In another preferred embodiment, the elastin-like fibers are woven into a diamond arrangement. In a preferred embodiment, the collagen-like fibers are arranged in a zig-zag, crimpled, or wavy configurations. In another preferred embodiment, the collagen-like fibers are simply loose or limp without preferred undulation. In at least one embodiment, the wavy configurations (e.g., the undulations, zig-zags, or crimps) are normal to the plane of the fabric. In a preferred embodiment, the wavy configurations are parallel to the plane of the fabric. In a preferred embodiment, the interwoven fabrics are then layered together, for example, in 2 to 200 layers, with the collagen-like fiber remaining loose.

In various embodiments, fibers of different mechanical (e.g., elastic) moduli may be used. In a preferred embodiment, two or more fibers are networked together by weaving, knitting, or other fiber manipulation system known to those skilled in the art. In one embodiment, the lower modulus fibers are formed into an elastic fabric, and successively higher moduli fibers are formed into an elastic fabric with successively higher moduli. In a preferred embodiment, the lowest moduli fibers are woven into a square arrangement. In a preferred embodiment, the lowest moduli fibers are woven into a diamond arrangement. The fabrics are then layered together, for example, in 2 to 200 layers with the successively higher moduli fabrics remaining successively loose. In a preferred embodiment, the higher moduli fibers are arranged in a zig-zag, crimped, or wavy configurations. In a preferred embodiment, the fabrics are annealed or fixed together. In a preferred embodiment, the higher moduli fibers are simply loose or limp without preferred undulation. In at least one embodiment, the wavy configurations (e.g., the undulations, zig-zags, or crimps) are normal to the plane of the fabric. In a preferred embodiment, the wavy configurations are parallel to the plane of the fabric.

In a preferred embodiment, the fibers are interwoven or interlocking to form an interpenetrating network comprising a fabric. In a preferred embodiment, the lowest moduli fibers are woven into a square arrangement. In another preferred embodiment, the lowest moduli fibers are woven into a diamond arrangement. In a preferred embodiment, the higher moduli fibers are arranged in a zig-zag, crimpled, or wavy configurations. In a preferred embodiment, the fabrics are annealed or fixed together. In another preferred embodiment, the higher moduli fibers are simply loose or limp without preferred undulation. In one embodiment, the wavy configurations (e.g., the undulations, zig-zags, or crimps) are normal to the plane of the fabric. In a preferred embodiment, the wavy configurations are parallel to the plane of the fabric. In a preferred embodiment, the interwoven fabrics are then layered together, for example in 2 to 200 layers, with the higher moduli fibers remaining loose.

In another embodiment, fiber networks comprised of elastin-like fibers and collagen-like fibers are formed in molds or printed. In a preferred embodiment, molds are engraved in a square relief pattern. Elastin-like networks are formed within and released from the molds to form an elastin-like network. In another preferred embodiment, the molds are engraved in a diamond relief pattern. The elastin-like networks are formed within and released from the molds to form the elastin-like network. In another preferred embodiment, the elastin-like networks are printed into a square network. In another preferred embodiment, the elastin-like networks are printed into a diamond network. In a preferred embodiment, the collagen-like fibers are interwoven into the elastin-like network. In a preferred embodiment, the collagen-like fibers are simply loose or limp without preferred undulation. In one embodiment, the undulations, zig-zags, crimps, or waviness are normal to the plane of the fabric. In a preferred embodiment, the undulations are parallel to the plane of the fabric. In a preferred embodiment, the collagen-like fibers are also formed into a network and joined (e.g., annealed, embossed, etc.) together with elastin-like fibers. In a preferred embodiment, the collagen-like fibers are simply loose or limp without preferred undulation. In one embodiment, the wavy configurations (e.g., the undulations, zig-zags, or crimps) are normal to the plane of the fabric. In a preferred embodiment, the wavy configurations are parallel to the plane of the fabric. In a preferred embodiment, the interwoven fabrics comprising one or more networks are then layered together, for example, in 2 to 200 layers, with the collagen-like fiber remaining loose. In a preferred embodiment, the interwoven fabrics comprising one or more networks are then annealed together, for example, in 2 to 200 layers, with the collagen-like fiber remaining loose. In a preferred embodiment, the collagen-like fibers are incorporated within the mold with a fiber length greater than the greater of the length or width of the mold. In one embodiment, the collagen-like fibers are incorporated loosely within the mold. In another embodiment, the collagen-like fibers are incorporated within the plane of the elastin-like network in a zig-zag, wavy, crimped, undulating, or aperiodic fashion. Other network forming options remain within the scope of the disclosure as known or apparent by those skilled in the art including, but not limited to, blow molding, extrusion, etc.

In another embodiment, fiber networks comprised of fibers of different mechanical (e.g., elastic) moduli are formed in molds or printed. In a preferred embodiment, molds are engraved in a square relief pattern. The lowest elastic modulus networks are formed within and released from the molds to form the lowest elastic modulus network. In another preferred embodiment, the molds are engraved in a diamond relief pattern. The lowest elastic modulus networks are formed within and released from the molds to form the lowest elastic modulus network. In another preferred embodiment, the lowest elastic modulus networks are printed into a square network. In another preferred embodiment, the lowest elastic modulus networks are printed into a diamond network. In a preferred embodiment, one or a plurality of higher elastic modulus fibers are interwoven into the lowest elastic modulus network. In a preferred embodiment, the higher elastic moduli fibers are simply loose or limp without preferred undulation. In one embodiment the undulations, zig-zags, crimps, or waviness are normal to the plane of the fabric. In a preferred embodiment, the undulations are parallel to the plane of the fabric. In a preferred embodiment, higher elastic moduli fibers are also formed into a network and joined (e.g., annealed, embossed, etc.) together with lower elastic moduli fibers. In a preferred embodiment, the higher elastic moduli fibers are simply loose or limp without preferred undulation. In one embodiment, the wavy configurations (e.g. the undulations, zig-zags, or crimps) are normal to the plane of the fabric. In a preferred embodiment, the wavy configurations are parallel to the plane of the fabric. In a preferred embodiment, the interwoven fabrics comprising one or more networks are then layered together, for example, in 2 to 200 layers, with the higher elastic moduli fibers remaining loose. In a preferred embodiment, the interwoven fabrics comprising one or more networks are then annealed together, for example, in 2 to 200 layers, with the higher elastic moduli fiber remaining loose. In a preferred embodiment, the higher elastic moduli fibers are incorporated within the mold with a fiber length greater than the greater of the length or width of the mold. In one embodiment, the higher elastic moduli fibers are incorporated loosely within the mold. In another embodiment, the higher elastic moduli fibers are incorporated within the plane of the lowest elastic modulus network in a zig-zag, wavy, crimped, undulating, or aperiodic fashion. Other network forming options remain within the scope of the disclosure as known or determined by those skilled in the art including, but not limited to, blow molding, extrusion, etc.

In another embodiment, elastin-mimetic material is formed into a sheet, film, or membrane. Holes with a diameter exceeding 10, 20, 30, 40, 50, 60, 70, 80, 90, 100, 110, 120, 130, 140, 150 micrometers are punctured in the membrane and collagen-like fibers are threaded therethrough.

In a preferred embodiment, two of more fibers are arranged into a linear suture. In a preferred embodiment, the central fiber is elastin-like and the one or more collagen-like fibers surround the central fiber and are loose. In another preferred embodiment, the central fiber is elastin-like and the one or more collagen-like fibers surround the central fiber and are woven into a loose fitting helical sheath. In a preferred embodiment, one or more sutures using such fibers are woven together into a weave or fabric. In yet another preferred embodiment, the central fiber is elastin-like and hollow. Within the hollow fiber, one or more collagen-like fibers reside with lengths greater than the length of the elastin-like fiber. In a preferred embodiment, one or more sutures using such fibers are woven together into a weave or fabric.

In a preferred embodiment, two or more fibers are arranged into a linear suture. In a preferred embodiment, the central fiber has a lower elastic modulus and the one or more higher elastic modulus fibers surround the central fiber and are loose. In another preferred embodiment, the central fiber has a lower elastic modulus and the one or more higher elastic modulus fibers surround the central fiber and are woven into a loose fitting (perhaps helical) sheath. In a preferred embodiment, one or more sutures using such fibers are woven together into a weave or fabric. In yet another preferred embodiment, the central fiber has a lower elastic modulus and is hollow. Within the hollow fiber, one or more higher elastic modulus fibers reside with lengths greater than the length of the lower elastic modulus fiber. In a preferred embodiment, one or more sutures using such fibers are woven together into a weave or fabric.

In a preferred embodiment, the fibers will be networked together auxetically such that when a tensile stress is applied, the mesh sling will increase in transverse direction. This will prevent the fiber from bunching up and increasing the risk of erosion by further exacerbating the mechanical strength of the synthetic mesh.

Additional preferred embodiments are disclosed in the following example.

### Example 1

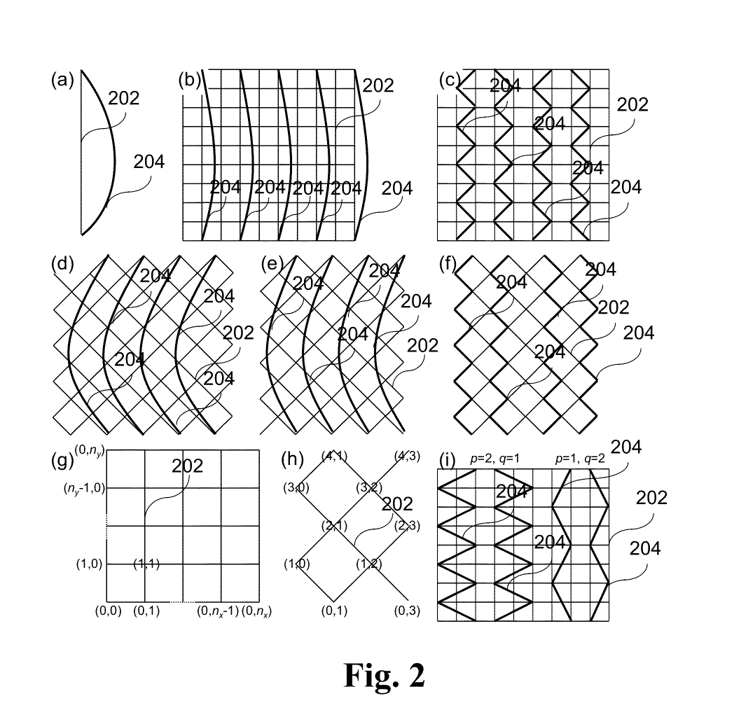

This example considers mesh composed of elastin-like fibers into which collagen-like fibers are woven and anchored at terminal junctions (see FIG. 2). FIG. 2 provides pictorial diagrams illustrating spatial arrangements of elastin 202 (thin) and collagen 204 (thick) fibers in (a) 1D and 2D with (b) elastin 202 in a square lattice and loose collagen 204, (c) elastin 202 in a square lattice and collagen 204 in a zig-zag pattern (p=q=1), (d) elastin 202 in a narrow diamond lattice and loose collagen 204, (e) elastin 202 in a wide diamond lattice and loose collagen 204, and (f) elastin 202 in a narrow diamond lattice and collagen 204 in a zig-zag arrangement (p=q=1). The nodes are numbered as ordered pairs (i, j) denoting row and column position, respectively, for (g) square and (h) diamond lattices. Crimped or zig-zag collagen pathways are shown (i) with p=2 and q=1 (left) and p=1 and q=2 (right).

In FIG. 2, each intersection of the fibers at which force is transferred is a static frictionless node at which Newton's first law holds. Each node is designated with indices i and j representing row position in the x-direction and column position in the y-direction, respectively. Because polymers are linearly elastic until their yield points, each fiber intersecting the node is modeled using as a non-yielding Hookian spring with a spring constant of EA/l^{o} for elastin-like fibers and E_{c}A_{c}/l_{c}^{o} for collagen-like fibers, were E and E_{c} are elastic moduli, A and A_{c} are the (composite) cross-sectional areas, and l^{o} and l_{c}^{o} are the vertical distances between nodes bounding fiber segments. Under linear elasticity, the force balances in x and y directions decouple. FIG. 2 shows the square and diamond lattices considered for the elastin network 202 along with the curvilinear and zig-zag collagen fiber arrangements 204. The elastin-like nodes are immediately adjacent, whereas the collagen-like nodal network may not be adjacent. The first row of nodes is anchored at fixed positions and labeled as i=0 so that intervals and node numbering directly correspond. The final row of nodes (at i=n_{y}, where n_{y} is the number of intervals or the node number in the y direction) experiences a force Fⱼ, the sum of which (Fₒᵥ=ΣⱼFⱼ) is the total force applied to the mesh to induce a specific deformation. These respective conditions represent a hard tissue anchor and organ force, for example. This arrangement was selected because POP and UI mesh is often anchored to stiff or hard tissue such as Cooper's ligament or bone on one end and sustain organ load from pelvic organs or the urethrovesicle junction (UVJ or bladder neck).

First, an analysis for a 1D mesh or individual fibers is developed, where only two spring forces pull at each node. This is representative of a multiple fiber suture, for example. Newton's first law for each elastin-like node gives

(EA/l^{o})[(yᵢ₊₁−yᵢ)−(yᵢ₊₁^{o}−yᵢ^{o})]+(EA/l^{o})[(yᵢ₋₁−yᵢ)−(yᵢ₋₁^{o}−yᵢ^{o})]=0, (1)

where yᵢ is the vertical position of node i with the super script o representing the initial pre stressed positions. This equation simplifies to

2yᵢ−yᵢ₊₁−yᵢ₋₁=0 (2)

which indicates that yᵢ is simply the average of the two adjacent nodal positions.

Arrangement of Eq. 2 for all nodes except the first (set to zero) and final (fixed displacement) rows in matrix form (AY=B) finds a Laplacian tridiagonal coefficient matrix with diagonal entries representing the degree of nodal connectivity (here 2 because each node joins two fibers) and off-diagonal elements representing adjacency valued at −1. B is an otherwise empty column vector with an entry of L^{o}+Δu in row i, only if that node connects to the final row of nodes, where L^{o} (=n_{y}l^{o}) is the initial total vertical length of the fiber, Δu is the imposed deformation of the entire fiber, and n_{y} is the number of nodes or intervals in the y-direction. Inversion determines that the last row entries of Y (corresponding to the next to last row of nodes) to be yᵢ₋₁=(L^{o}+Δu)(n_{y}−1)/n_{y}. The final node force balance for an elastin only network becomes

(EA/l^{o})[(yᵢ₋₁−yᵢ)−(yᵢ₋₁^{o}−yᵢ^{o})]+Fᵢ=0, (3)

such that

Fₒᵥ/EA=Fᵢ/EA=(yᵢ−yᵢ₋₁)/l^{o}−1=Δu/L^{o} (4)

in scaled (nondimensionalized) form. This result may have been expected with Hooke's law because Δu/L^{o} is the strain and Fᵢ/A is the stress by definition. However, inclusion of a spanning collagen-like fiber that is initially limp modifies the final row force balance such that

(EA/l^{o})[(yᵢ₋₁−yᵢ)−(yᵢ₋₁^{o}−yᵢ^{o})]+(E_{c}A_{c}/L_{c}^{o})[(yₒ−yᵢ)+L_{c}^{o})]H(L^{o}+Δu−L_{c}^{o})+Fᵢ=0, (5)

where H( ) is the Heavyside function valued at unity when the argument is positive definite and zero otherwise to indicate that the collagen only contributes to the force balance when the elastin fiber is as long as the collagen fiber. Then

Fₒᵥ/EA=Fᵢ/EA=Δu/L^{o}+(E_{c}A_{c}L^{o}/EAL_{c}^{o})[(L^{o}+Δu−L_{c}^{o})/L^{o}]H(L^{o}+Δu−L_{c}^{o}). (6a)

The dimensionless number E_{c}A_{c}L^{o}/EAL_{c}^{o} immediately appears representing the ratio of collagen to elastin-like fiber properties. This solution also defines a critical displacement, Δu*=L_{c}^{o}−L^{o}, which indicates the offset on force displacement curves before the collagen-like fibers engage to sharply increase the slope. This displacement is a tunable parameter determined by initial fiber arrangement. Rewriting this equation in terms of Δu* yields

Fₒᵥ/EA=Δu/L^{o}+(E_{c}A_{c}L^{o}/EAL_{c}^{o})H(Δu/L^{o}−Δu*/L^{o})[Δu/L^{o}−Δu*/L_{c}^{o}]. (6b)

which will be compared to similar expressions for the mesh.

2D arrangements of elastin-like fibers can adopt square or diamond lattices. Both arrangements are found in commercially available mesh. In a square arrangement (see FIGS. 2b and c), each node can have 2-4 elastin connections. A force balance at a fully internal node with four connections yields

(EA/l^{o})[(yᵢ₊₁ⱼ−yᵢⱼ)−(yᵢ₊₁ⱼ^{o}−yᵢⱼ^{o})]+(EA/l^{o})[(yᵢ₋₁ⱼ−yᵢⱼ)−(yᵢ₋₁ⱼ^{o}−yᵢⱼ^{o})]+(EA/l^{o})[(yᵢⱼ₊₁−yᵢⱼ)−(yᵢⱼ₋₁^{o}−yᵢⱼ^{o})]+(EA/l^{o})[(yᵢⱼ₋₁−yᵢⱼ)−(yᵢⱼ₋₁^{o}−yᵢⱼ^{o})]=0, (7)

which simplifies to

4yᵢⱼ−(yᵢ₊₁ⱼ+yᵢ₋₁ⱼ+yᵢⱼ₊₁+yᵢⱼ₋₁)=0, (8)

leaving yᵢⱼ again as the average of the positions of the adjacent nodes. On the edge of the network, nodes with only three connections have force balances in the form of

3yᵢⱼ−(yᵢ₊₁ⱼ+yᵢ₋₁ⱼ+yᵢⱼ±1)−0, (9)

with the plus and minus entries for the first (j=0) and final (j=nₓ, where nₓ is the number of nodes in the x-direction) column of nodes, respectively. With yₙ_{_{y}}ⱼ=L^{o}+Δu as the driving force, Eqs. 8-9 can also be rearranged into a Laplacian pentadiagonal coefficient matrix with two additional diagonal adjacency entries offset from the primary degree of connectivity diagonal by nₓ−1. Inversion again leads to displacements of the next to final row of yᵢ=(L^{o}+Δu)(n_{y}−1)/n_{y}, because a constant displacement has been imposed. The force balance on the final row of nodes determines the required force applied. The terminal row force balance becomes

Fᵢⱼ+(EA/l^{o})[(yᵢ₋₁ⱼ−yᵢⱼ)−(yᵢ₋₁ⱼ^{o}−yᵢⱼ^{o})]+(EA/l^{o})[(yᵢⱼ₊₁−yᵢⱼ)−(yᵢⱼ₊₁^{o}−yᵢⱼ^{o})]+(EA/l^{o})[(yᵢⱼ₋₁−yᵢⱼ)−(yᵢⱼ₋₁^{o}−yᵢⱼ^{o})]=0, (10)

which for both central and edge positions becomes

Fᵢⱼ/EA=yᵢⱼ/l^{o}−yᵢ₋₁ⱼ/l^{o}−1, (11)

recognizing that each displacement in a given row is equivalent. The total force required (Fₒᵥ=Σⱼ₌₀ⁿ^{ₓ}Fᵢ₍₌ₙ_{_{y}}₎ⱼ) then becomes

Fₒᵥ/EA=nₑₛΔu/L^{o}, (12)

which differs from the 1D solution only in its accounting for the number of elastin-like strands in the network.

A 2D diamond arrangement of an elastin-like network can have 1-4 forces acting at each node depending on its connectivity. In this arrangement, every other ij combination corresponds to a connected node. A fully connected node becomes

(EA/l^{o})[(yᵢ₋₁ⱼ₋₁−yᵢⱼ)−(yᵢ₋₁ⱼ₋₁^{o}−yᵢⱼ^{o})]+(EA/l^{o})[(yᵢ₋₁ⱼ₊₁−yᵢⱼ)−(yᵢ₋₁ⱼ₊₁^{o}−yᵢⱼ^{o})]+(EA/l^{o})[(yᵢ₊₁ⱼ₋₁−yᵢⱼ)−(yᵢ₊₁ⱼ₊₁^{o}−yᵢⱼ^{o})]+(EA/l^{o})[(yᵢ₊₁ⱼ₊−yᵢⱼ)−(yᵢ₊₁ⱼ₊₁^{o}−yᵢⱼ^{o})]=0, (13)

which simplifies to

4yᵢⱼ−(yᵢ₋₁ⱼ₋₁+yᵢ₋₁ⱼ₊₁+yᵢ₊₁ⱼ₋₁+yᵢ₊₁ⱼ₊₁)=0, (14)

where again l^{o} corresponds to the initial vertical distance between nodes in any given network (not the diagonal node-to-node distance). On the edges,

2yᵢⱼ−(y_{i-1j±1}+y_{i+1j±1})=0 (15)

with plus and minus entries corresponding to the first (j=0) and final (j=nₓ) column of nodes, respectively. Fixing the displacement of the final row of nodes to L^{o}+Δu, assembling a Laplacian or Kirchhoff connectivity matrix and a vector containing nonzero entries for any node connected to a node with i=n_{y}, and then inverting again leads to y_{ny-1}=(L^{o}+Δu)(n_{y}−1)/n_{y}. The final row forces balances become

Fᵢⱼ+(EA/l^{o})[(yᵢ₋₁ⱼ₋₁−yᵢⱼ)−(yᵢ₋₁ⱼ₋₁^{o}−yᵢⱼ^{o})]+(EA/l^{o})[(yᵢ₋₁ⱼ₊₁−yᵢⱼ)−(yᵢ₋₁ⱼ₊₁^{o}−yᵢⱼ^{o})]=0 (16)

for a central node, which simplifies to

Fᵢⱼ/EA=2yᵢⱼ/l^{o}−(yᵢ₋₁ⱼ₋₁+yᵢ₋₁ⱼ₊₁)/l^{o}−2, (17)

and for an edge node with only one connection to

Fᵢⱼ/EA=yᵢⱼ/l^{o}−y_{i-1j±1}/l^{o}−1. (18)

This example considers both narrow terminal row configuration where all terminal nodes have two connections and wide terminal row configuration where the first and last column nodes have only one connection. In both cases, following a similar solution strategy as above finds yᵢ=(L^{o}+Δu)(n_{y}−1)/n_{y}, such that

Fₒᵥ/EA=nₑₛΔu/L^{o}. (19)

Note that nₑₛ is the number of elastin fibers that cross any internodal horizontal plane. This shows that the fiber arrangement is unimportant in terms of uniaxial applied stresses but the number of elastin strands is important.

One of the purposes of this example is to evaluate the role of dual strand lattices. This example now evaluates initially loose collagen strands spanning the full length of the elastin lattice. Because these higher modulus fibers connect only at the initial and final rows, the elastin lattice displacements do not change. The terminal row force balance, in contrast, sustains additional terms wherever a collagen-like fiber links in.

Fⱼ+(EA/l^{o})[(yᵢ₋₁ⱼ−yᵢⱼ)−(yᵢ₋₁ⱼ^{o}−yᵢⱼ^{o})]+(E_{c}A_{c}/L_{c}^{o})[(yₒⱼ−yᵢⱼ)+L_{c}^{o})]H(L^{o}+Δu−L_{c}^{o})+(EA/l^{o})[(yᵢⱼ₊₁−yᵢⱼ)−(yᵢⱼ₊₁^{o}−yᵢⱼ^{o})]+(EA/l^{o})[(yᵢⱼ₋₁−yᵢⱼ)−(yᵢⱼ₋₁^{o}−yᵢⱼ^{o})]=0, (20)

which simplifies to

Fⱼ/EA=Δu/L^{o}+(E_{c}A_{c}/EA)[(L^{o}+Δu)/L_{c}^{o}−1]H(L^{o}+Δu−L_{c}^{o}) (21)

for both central and edge positions. Terminal nodes for which the collagen fibers do not connect retain their previous form (see Eq. 11). The overall applied force is then

Fₒᵥ/(nₑₛEA)=Δu/L^{o}+(n_{cs}E_{c}A_{c}L^{o}/nₑₛEAL_{c}^{o})H[Δu/L^{o}−Δu*/L^{o}][Δu/L^{o}−Δu*/L^{o}]. (22)

Careful evaluation of the force balance for elastin-like diamond lattices, Eq. 16, with the addition of (E_{c}A_{c}/L_{c}^{o})[(yₒⱼ−yᵢⱼ)+L_{c}^{o})]H(L^{o}+Δu−L_{c}^{o}) finds an identical stress-strain relationship, which is expected because the square and diamond elastin-like lattices are identical in their stress-strain relationships.

Finally, this example considers the collagen-like fibers in zig-zag or crimped arrangements, where the fiber weaves around specific nodes until it becomes taut. Prior to becoming taut, the collagen-like fibers do not participate in the force balance because they are limp. At the moment they become taut, L_{c}^{o}=L^{o}[1+(p/q)²]^{1/2} and l_{c}^{o}=l^{o}[1+(p/q)²]^{1/2}, where p and q are the offset between consecutive collagen-like fiber nodes in the x and y directions, respectively (see FIG. 2i). Even then, the collagen-like fibers do not significantly participate in the y components of the force balance so long as the collagen-like fiber modulus significantly exceeds that of the elastin-like fibers; they do, however, participate in the x components. Only when the elastin-like fibers have stretched to the initial length of the collagen-like fibers do they participate in the stress balance. Then, the force balance on the square lattice is

$$( EA / {l}^{°} ) [ ( {y}_{i + 1 j} - {y}_{ij} ) - ( {y}_{i + 1 j}^{°} - {y}_{ij}^{°} ) ] + ( EA / {l}^{°} ) [ ( {y}_{i - 1 j} - {y}_{ij} ) - ( {y}_{i - 1 j}^{°} - {y}_{ij}^{°} ) ] + ( EA / {l}^{°} ) [ ( {y}_{ij + 1} - {y}_{ij} ) - ( {y}_{ij + 1}^{°} - {y}_{ij}^{°} ) ] + ( EA / {l}^{°} ) [ ( {y}_{ij - 1} - {y}_{ij} ) - ( {y}_{ij - 1}^{°} - {y}_{ij}^{°} ) ] + ( {E}_{c} {A}_{c} / {l}_{c}^{°} ) [ ( {y}_{i - qj \pm p} - {y}_{ij} ) + ( {y}_{i - qj \pm p}^{°} - {y}_{ij}^{°} ) ] H ( {L}^{°} + Δ u - {L}_{c}^{°} ) + ( {E}_{c} {A}_{c} / {l}_{c}^{°} ) [ ( {y}_{i + qj \pm p} - {y}_{ij} ) + ( {y}_{i + qj \pm p}^{°} - {y}_{ij}^{°} ) ] H ( {L}^{°} + Δ u - {L}_{c}^{°} ) = 0 , \qquad ( 23 )$$

where m is the number of nodal row intervals between collagen-like fiber junctions and n is the number of nodal column intervals between collagen-like fiber junctions. This simplifies to

4yᵢⱼ−(yᵢ₊₁ⱼ+yᵢ₋₁ⱼ+yᵢⱼ₊₁+yᵢⱼ₋₁)+l^{o}/EA[2(E_{c}A_{c}/l_{c}^{o})yᵢⱼ−E_{c}A_{c}/l_{c}^{o}(y_{i-qj±p}+y_{i+qj±p})]H(L^{o}+Δu−L_{c}^{o})=0. (24)

On the edge of the lattice, nodes with only three connections have force balances in the form of

3yᵢⱼ−(yᵢ₊₁ⱼ+yᵢ₋₁ⱼ+y_{ij±1})+l^{o}/EA[2(E_{c}A_{c}/l_{c}^{o})yᵢⱼ−E_{c}A_{c}/l_{c}^{o}(y_{i-qj±p}+y_{i+qj±p})]H(L^{o}+Δu−L_{c}^{o})=0, (25)

with the plus and minus entries for the first (j=0) and final (j=nₓ) column of nodes, respectively. When rewritten for all nodes in matrix form (AY=B), the system of equations takes the form

([A]ₑₗₐₛₜᵢₙ+(E_{c}A_{c}/EA)(l^{o}/l_{c}^{o})[A]_{collagen})Y−([B]ₑₗₐₛₜᵢₙ+(E_{c}A_{c}/EA)(l^{o}/l_{c}^{o})[B]_{collagen}), (26)

where [A]ₑₗₐₛₜᵢₙ is identical to the pentadiagonal Kirkhoffs connectivity matrix presented above for elastin-like fibers, [A]_{collagen} is a sparse matrix with nonzero diagonal matrix elements (here equal to two) where the collagen-like fiber junctions with the node and off diagonal matrix elements (equal to −1) corresponding to the adjacency between nodes defined by the collagen-like path, [B]ₑₗₐₛₜᵢₙ is the column vector with nonzero entries of L^{o}+Δu where node ij is mathematically adjacent to a terminal row node as defined by the elastin network, and [B]_{collagen} is the column vector with nonzero entries of L^{o}+Δu where node ij is mathematically adjacent to a terminal row node as defined by the collagen network. Inversion and solution again finds the next to final row displacements to be y_{ny-qj}=(L^{o}+Δu)(n_{y}−q)/n_{y} with all entries in a particular row being equivalent. For symmetric collagen arrangements, this is always the case. The final node force balance for an elastin-only network becomes

Fᵢ=EA/l^{o}[(yᵢⱼ−yᵢ₋₁ⱼ)−l^{o}]+(E_{c}A_{c}/l_{c}^{o})[(yᵢⱼ−y_{i-qj±p})−l_{c}^{o}]H(L^{o}+Δu−L_{c}^{o}), (27)

which can be simplified to

Fᵢ/EA=Δu/L^{o}+(E_{c}A_{c}/EA)[(Δu+L^{o})/l_{c}^{o}·q/n_{y}−1)]H(L^{o}+Δu−L_{c}^{o}). (28)

The vertical length between collagen nodes can be determined from the total length of the collagen strand as l_{c}^{o}=L_{c}^{o}q/n_{y}. Substitution then finds

Fᵢ/EA=Δu/L^{o}+(E_{c}A_{c}L^{o}/EAL_{c}^{o})[Δu/L^{o}−Δu*/L^{o}]H(Δu/L^{o}−Δu*/L^{o}) (29)

so that)

Fₒᵥ/(nₑₛEA)=Δu/L^{o}+(n_{cs}E_{c}A_{c}L^{o}/nₑₛEAL_{c}^{o})[Δu/L^{o}−Δu*/L^{o}]H(Δu/L^{o}−Δu*/L^{o}). (30)

This solution is identical to Eq. 22, with the single caveat that a clear relationship between L_{c}^{o} and L^{o} or between l_{c}^{o} and l^{o} is now obtained.

The above results can be generalized to a continuum of fibers (e.g. three or more fibers in the mesh by including several collagen-like strands that span the mesh as

(EA/l^{o})[(yᵢ₋₁−yᵢ)−(yᵢ₋₁^{o}−yᵢ^{o})]+Σₖ(EₖAₖ/Lₖ^{o})[(yₒ−yᵢ)+Lₖ^{o})]H(L^{o}+Δu−Lₖ^{o})+Fᵢ+(EA/l^{o})[(yᵢⱼ₊₁−yᵢⱼ)−(yᵢⱼ₊₁^{o}−yᵢⱼ^{o})]+(EA/l^{o})[(yᵢⱼ₋₁−yᵢⱼ)−(yᵢⱼ₋₁^{o}−yᵢⱼ^{o})]=0, (31)

where each collagen-like strand is denoted by index k. Simplifying as above yields

Fₒᵥ/(nₑₛEA)=Δu/L^{o}+Σₖ(nₖₛEₖAₖL^{o}/nₑₛEALₖ^{o})[Δu/L^{o}−Δuₖ*/L^{o}]H(Δu/L^{o}−Δuₖ*/L^{o}) (32)

This shows that there are now a series of dimensionless groups for each term. As before, Δuₖ*/L^{o} represents the critical strain for strands k of which there are nₖₛ, and the second dimensionless group represents the ratio of material and geometric properties of strand k to that of elastin, where Eₖ and Aₖ represent the elastic modulus and cross-sectional area of strand k.

Results

This example uses the spring network model to design and evaluate dual fiber represents elastin-like fibers of low elastic modulus, while the second fiber represents collagen-like fibers with higher moduli. Both types of fibers independently span (or percolate across) the entire length of the tissue implant. If the higher modulus fibers do not percolate across the entire mesh, then lower moduli bridges between higher moduli fibers sustain a majority of the strain until the network begins to yield as they fail and the behavior of the network is the controlled almost exclusively by the low modulus fibers. This is a trivial case and not considered further herein.

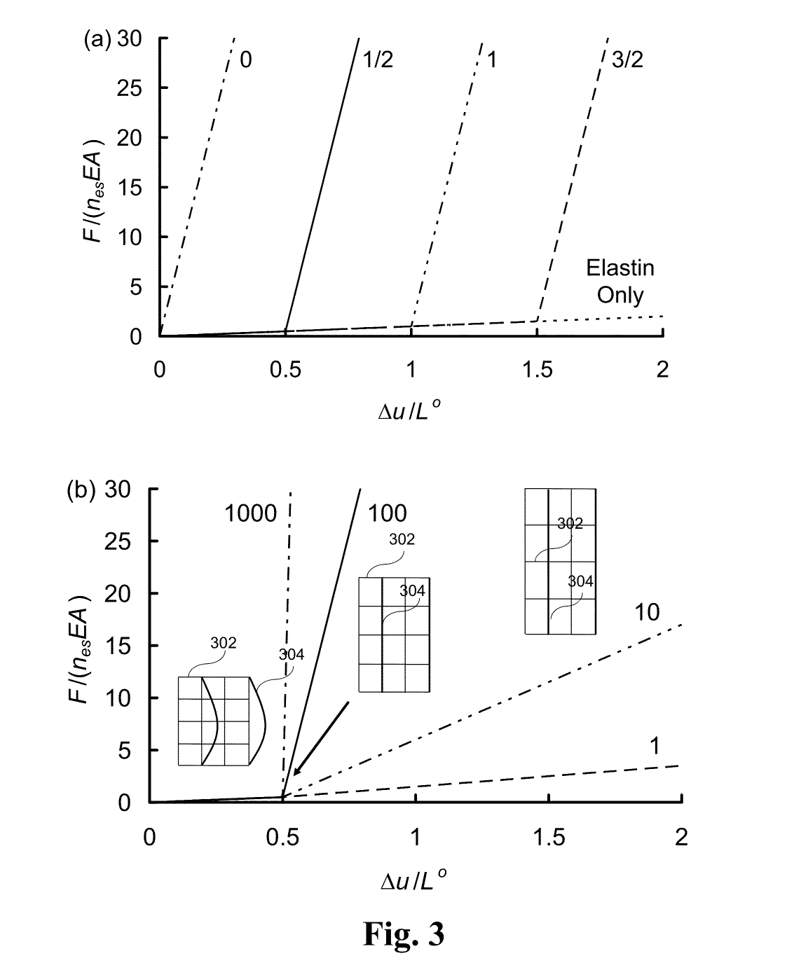

FIG. 3 presents scaled force-displacement curves that summarize the key results of the analysis as encapsulated in Eqs. 22 and 30, which are identical. The force is scaled on the product of the number, elastic modulus, and cross-sectional area of the elastin-like strands, which is equivalent to scaling the stress on the elastic modulus of the elastin where the stress is the ratio of the force applied and the total cross-sectional area of all elastin strands. The displacement is scaled on the initial length, which is by definition the strain. The curves typically show two distinct slopes. The first slope corresponds to strain of only elastin fibers, while the second slope corresponds to strain of both fibers, but since the collagen-like fibers typically have significantly higher elastic moduli, the second slope is controlled predominantly by the collagen-like fibers.

Two dimensionless groups govern the family of stress-strain curves. Indeed, the curves are scaled to show that the entire solution set may be represented by two families of curves defined by these two groups in FIG. 3. The first dimensionless group is the critical strain, Δu*/L^{o}. This dimensionless group represents the overhang or initial looseness of the collagen-like fibers relative to the elastin-like fibers. For example, if the collagen-like fibers are 50% longer than the initial length of the elastin-like fibers, then Δu*/L^{o}=½. FIG. 3a shows that this group determines the transition between the two slopes. When the network strain is less than Δu*/L^{o}, the collagen fibers remain limp and the elastin-like fibers bear all of the stress. As the network strain exceeds Δu*/L^{o}, the collagen-like fibers engage and bear a majority, if not all, of the load. FIG. 3a also shows that if Δu*/L^{o} falls to zero, then the collagen-like fibers engage immediately and that the mechanical response of the system is governed almost exclusively by the collagen-like fibers. This latter case represents the current design of most polymeric mesh; see FIG. 1.

The second dimensionless group represents the ratio of the material properties of the collagen-like fibers 304 to the elastin-like fibers 302, n_{cs}E_{c}A_{c}L^{o}/nₑₛEAL_{c}^{o}. FIG. 3b shows that as this ratio increases, the slope of the linear region rises dramatically. So long as this dimension ratio exceeds unity, the stress-strain curves have two distinct slopes and the collagen-like fibers dominate the behavior of the linear region. Several factors participate in this group including the elastic moduli. Typical moduli of collagen fibers are in the range of 100 MPa to 1 GPa, whereas typical values for elastin are in the range of 0.1-1 MPa, suggesting that dimensionless ratios on the order of 100-1000 are not unexpected.

The analysis considers two distinct arrangements of the elastin-like fiber mesh, namely square and diamond arrangements (see FIG. 2). Remarkably, neither the orientation of the lattice nor the lattice spacing directly influences the stress-strain curves, only the number of spanning strands. (If the number of elastin strands 202 per cross section changes, then the orientation becomes influential.) Similarly, loose collagen strands and those arranged into crimped or zig-zag configurations also find an identical stress-strain curve. This remarkable result provides a significant degree of design flexibility and shows that the family of curves in FIG. 3 is consistent for all types of elastin-like 302 and collagen-like 304 fiber arrangements.

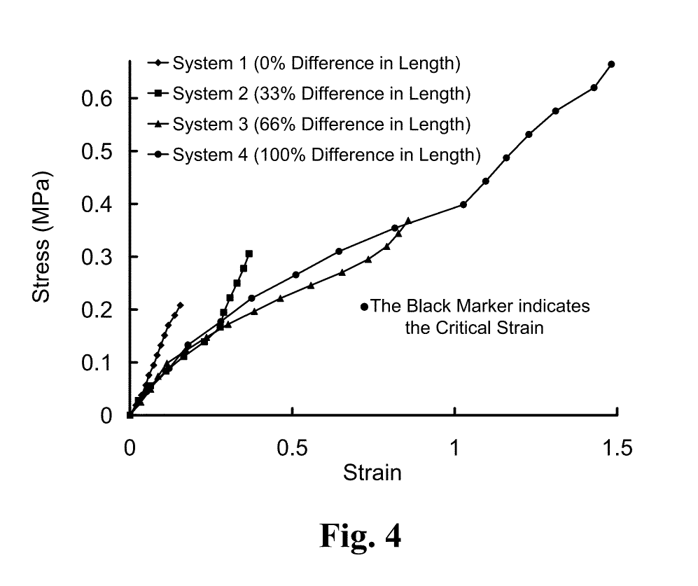

To evaluate this analysis quantitatively, both 1D and 2D fiber arrangements are generated and network elastic properties are measured. FIG. 4 evaluates the role of the critical stress for two fibers in 1D, which is representative of a multifiber suture, for example. The curves in the figure confirm that increasing the collagen-like fiber overhang does indeed delay the onset of the linear region as predicted by Eq. 22 (with one horizontal node). As the number of fibers increases, the slope of the linear region increases as predicted by the above analysis.

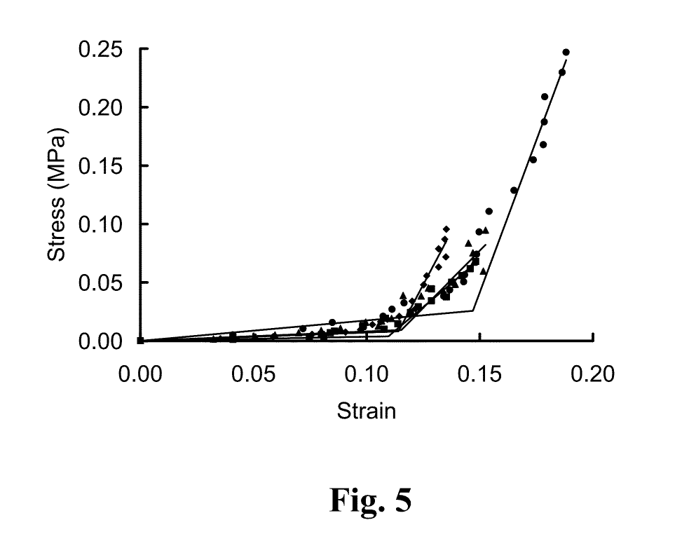

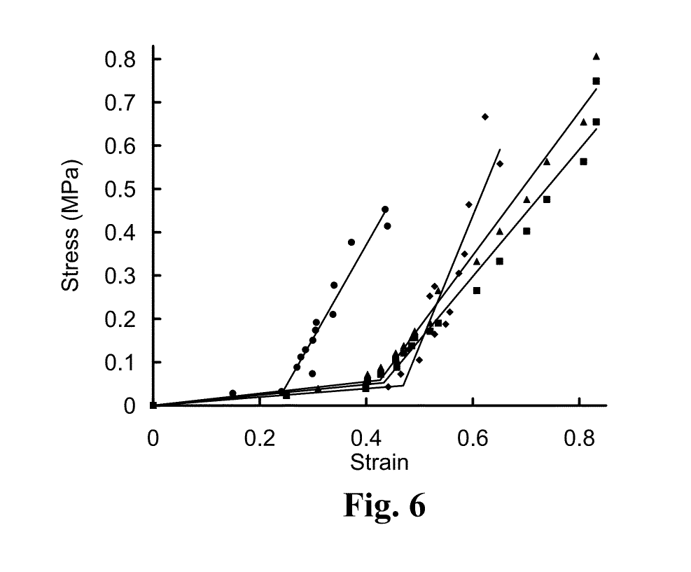

To aid in the design of fiber networks that most accurately match that of human tissue, the elastic properties of bovine fascia were measured. Rabbit (from the literature) and bovine tissue samples are readily available, whereas human cadaver tissue is less so. FIG. 5 shows several distinct stress-strain curves for bovine tissue. FIG. 6 shows several distinct stress-strain curves for Macaca monkey tissue. Both show that a two slope model fits reasonably well suggestive of collagen and elastin like fibers, though the exact values differ between species and between samples of the same species.

To directly compare the stress-strain curves from the fascia with the network model, it is noted that there is a subtle distinction in the scaling. The network force is scaled on the total cross sectional area of the elastin fibers, whereas the force on the tissue is scaled on its cross-sectional area. These two definitions of area are distinct but related by

$$\frac{{σ}_{es}}{E} {φ}_{es} = \frac{F}{{n}_{es} EA} {φ}_{es} = \frac{F}{{EA}_{tissue}} = \frac{{σ}_{tissue}}{E} , \qquad ( 33 )$$

where φₑₛ=nₑₛA/Aₜᵢₛₛᵤₑ is the areal fraction of the elastin-like fibers. Even where the elastin fibers are closely packed together, the cross-sectional area of the fibers will always be less than that of the entire tissue and φₑₛ remains strictly less than unity, requiring that F/nₑₛEA>F/EAₜᵢₛₛᵤₑ. The second dimensionless groups can similarly be rewritten in terms of areal fractions as n_{cs}E_{c}A_{c}L^{o}/nₑₛEAL_{c}^{o}=φ_{cs}E_{c}L^{o}/φₑₛEL_{c}^{o}, where φ_{cs}=n_{cs}A/Aₜᵢₛₛᵤₑ is the areal fraction of the collagen-like fibers. Hence direct comparison between the above analysis and experiments only requires the addition of the two areal fractions.

Discussion

The need to make synthetic biomimetic tissue support is well recognized across a broad range of biomedical engineering specialties. Polymeric mesh for surgical treatment of UI and POP in particular have typically incorporated relatively stiff polymers including nylon, polyester, and polypropylene in laminate and networked structures. These polymers have elastic moduli similar to that of collagen, which is important because relatively stiff polymers remain essential to upgrading damaged fascia to withstand the quotidian stresses readily sustained by healthy fascia. However, the two slope behavior is also a well recognized attribute of connective tissue including ligaments, tendons, and fascia not typically designed into polymeric constructs for soft tissue engineering that cannot be achieved with one type of linear fiber alone.

FIG. 3 shows that a key to imitating this property of fascia is to select two fibers and allow the stiffer (or larger diameter) of the two fibers to remain limp until a critical strain. This strain is directly and easily controlled by tuning selection of the overhang of the stiffer fiber relative to the less stiff of the two fibers. Indeed, the untensioned nature (i.e., looseness or limpness) of the stiffer fiber is an important attribute. Nature achieves this by arranging collagen fibers into zig-zag patterns in well-organized tissue such as tendons or within loosely organized tissues by leaving the fibers limp enough to follow curvilinear profiles on length scales that exceed the fiber's persistence length. By integrating a stronger and weaker fiber together, the mesh maintains mechanical stiffness necessary to withstand external forces but also provides a less stressful environment for cells resting on the elastin-like fibers. Indeed, control of the local microstress environment may be vital to minimizing the tissue erosion associated with polymeric implants and tuning cell signaling. Cells cannot react sufficiently to respond to excessively sharp forces, leading to cellular damage and a commensurate immune response that weakens the mechanical strength of the tissue and its support for organ load.

A remarkable feature of the solutions above is that they depend on only two dimensionless groups, the critical strain and a ratio of material properties. This allows for remarkable flexibility in design of the mesh. A variety of distinct parameter values can achieve the same mesh response. For example, the slope of the linear region may be controlled by choosing a relatively strong elastic modulus, enhancing the areal fraction of the collagen-like fibers, by increasing the fiber diameter, or by a combination of two or more of these factors. Nature has implemented several of these factors in connective tissue constructs. For example, collagen fibers form into bundles and increase their effective area in regions of high stress. The only primary limitation on the selection of the critical strain, the other dimensionless group, is that it must lie below the yield point of the weaker polymer (for elastin, the yield point corresponds to ˜300% elongation). Indeed, perhaps one of several reasons connective tissue has a dual slope stress-strain curve is to prevent the weaker fibers like elastin and fibrin from exceeding their yield points under extreme stress.

To determine the values of the parameters in the above example, bovine tissue was used. The magnitude of variation in the mechanical properties of the fascia not only complicates design based on these results, but is likely one of the key reasons for the disparity in clinical outcomes, though other factors including surgeon skill and patient adherence to recovery protocols also likely have a significant role as well. FIGS. 5 and 6 show the elastic modulus in the toe region to be 0.09-0.14 MPa for monkey tissue and 0.04-0.18 for bovine tissue, which is not unexpected for elastin rich tissues. However, the elastic modulus in the linear region is only 1.1-5.2 MPa for bovine tissue and 1.4-3.1 MPa for monkey tissue, which remains much less than the elastic modulus of pure collagen, which is at least two orders of magnitude larger. The final equation above indicates that the slopes can be lower than expected only when the areal fraction of collagen is small. Indeed, from these measurements, 10% of the hydrated fascial volume is composed of collagen fibers.

Finally, the model is readily extendable to multiple fiber arrangements, which may be advantageous because multiple fiber arrangements may more precisely match the curvature of fascia's stress-strain curve (see FIGS. 1 and 5). Indeed, natural tissue is composed of a continuum of fibers both in composition or modulus and in diameter. To represent this case, Eq. 32 contains multiple terms, one for each type and diameter of fiber. Each term contains a unique critical strain and unique dimensionless material group. For example, a three fiber network containing elastin-like, weak collagen-like, and strong collagen-like fibers would have two collagen-like terms, where the dimensionless material group and critical strain of the strong collagen-like fiber must both exceed the respective groups for the weak collagen-like fiber. In practice, however, there is an engineering balance between including more fibers to more precisely represent the stress-strain curve of fascia and the manufacturing challenge of including them.

### Example 2

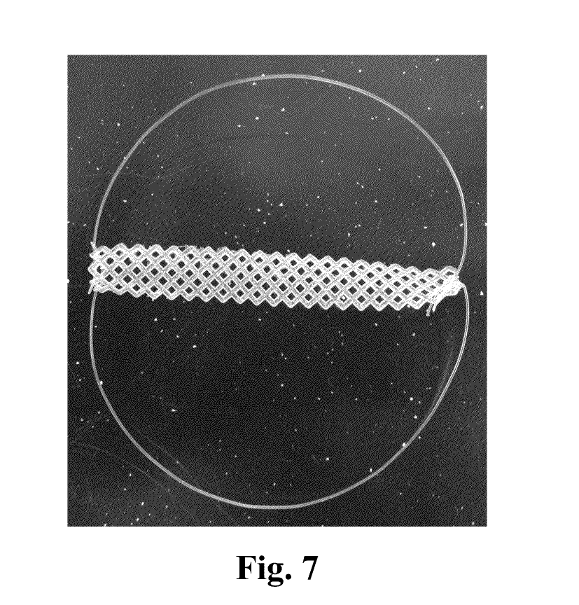

A dual fiber or dual material composite mesh was prepared from polydimethylsiloxane (PDMS) and polylactic acid (PLA), see FIG. 7. The PDMS has elastic properties similar to that of elastin, and PLA has a much higher modulus similar to collagen having a much higher tensile modulus than elastin. The PDMS was prepared by generating a mold in aluminum with groves in specific locations, filling the mold with PDMS to generate a fiber network, allowing the PDMS to cure and then removing the PDMS from the mold. The PLA fibers were secured at one end, woven through the mesh, and securing at the opposite end, all the while allowing PLA fiber to remain loose prior to tensioning the mesh.

From the foregoing, it will be appreciated that specific embodiments of the disclosure have been described herein for purposes of illustration, but that various modifications may be made without deviating from the spirit and scope of the disclosure.

Aspects described in the context of particular embodiments may be combined or eliminated with other embodiments. Further, although advantages associated with certain embodiments have been described in the context of those embodiments, other embodiments may also exhibit such advantages, and not all embodiments need necessarily exhibit such advantages to fall within the scope of the disclosure. Accordingly, the scope of the claimed invention is not limited except as by the appended claims.

The embodiments of the invention in which an exclusive property or privilege is claimed are defined as follows:

1. A material comprising two or more fibers, wherein each of the fibers has a mechanical modulus, wherein the mechanical modulus of at least one fiber is higher than the mechanical modulus of another fiber, and wherein the higher modulus fiber has a longer length than the lower modulus fiber.

2. The material of claim 1, wherein at least one of the fibers is a monofilament fiber.

3. The material of claim 1, wherein at least one of the fibers is a polyfilament fiber.

4. The material of claim 1, wherein the lower modulus fiber has an elastic modulus in the range of 0.1 to 10 MPa.

5. The material of claim 1, wherein the higher modulus fiber has an elastic modulus in the range of 1 to 10000 MPa.

6. The material of claim 5, wherein the higher modulus fiber is untensioned.

7. The material of claim 5, wherein the higher modulus fiber includes a wavy configuration.

8. The material of claim 1, wherein the two or more fibers are woven to form a network.

9. The material of claim 1, wherein the two or more fibers are knitted to form a network.

10. The material of claim 1, wherein at least one of the fibers is biodegradable.

11. The material of claim 1, wherein at least one of the fibers is not biodegradable.

12. The material of claim 1, wherein the two or more fibers are arranged to produce an auxetic material in which the width of the auxetic material expands instead of shrinks upon tensile stress.

13. The material of claim 1, wherein the two or more fibers are arranged to produce fabric sheets, and wherein 2 to 200 fabric sheets are layered together.

14. The material of claim 1, wherein the higher modulus fiber is collagen mimetic.

15. The material of claim 1, wherein the lower modulus fiber is elastin mimetic.

16. A suture, comprising:
two or more fibers,
wherein at least one of the fibers is elastin-like and has a lower elastic modulus than another fiber that is collagen-like and has a higher elastic modulus; and
wherein the higher modulus collagen-like fiber is longer than the lower modulus elastin-like fiber.

17. The suture of claim 16, wherein the collagen-like fiber surrounds the elastin-like fiber.

18. The suture of claim 16, wherein the collagen-like fiber is positioned within a hollow elastin-like fiber.

19. The suture of claim 16, wherein the at least one lower modulus fiber has an elastic modulus in the range of 0.1 to 10 MPa.

20. The suture of claim 16, wherein the at least one higher modulus fiber has an elastic modulus in the range of 1 to 10000 MPa.
